(19) United States
(12) Patent Application Publication
(10) Pub. No.: US 2022/0117786 A1
(43) Pub. Date: Apr. 21, 2022

(54) METHOD AND APPARATUS FOR TREATING OCULAR TISSUE

(71) Applicant: EXCELSIUS MEDICAL CO., LTD., TAINAN CITY (TW)

(72) Inventor: CHENG-HAO HUANG, TAINAN CITY (TW)

(21) Appl. No.: 17/074,860

(22) Filed: Oct. 20, 2020

(51) Int. Cl. A61F 9/008

(52) U.S. Cl. CPC A61F 9/00802; A61F 2009/00872; A61F 9/00836

(57) ABSTRACT

The present invention discloses a method and an apparatus for treating an ocular tissue. The method for treating the ocular tissue includes generating a femtosecond laser beam from a laser source; orientating the femtosecond laser beam toward the ocular tissue; and defining a target area in the ocular tissue using the femtosecond laser beam, wherein the target area contains a sharp-edge part and a to-be-removed part, in which the sharp-edge part has a minimum thickness being gradually reduced to zero and is ablated by the femtosecond laser beam while the target area is defined; and removing the to-be-removed part of the target area from the ocular tissue.

### BACKGROUND OF THE INVENTION

### 1. Technical Field

The present invention relates to a method and an apparatus for treating an ocular tissue.

### 2. Description of Related Art

For vision correction, a conventional solution using excimer laser to make changes in corneal curvature is known as laser vision correction (LVC). Among LVC-based procedures, the most commonly used is laser-assisted in situ keratomileusis (LASIK), taking up about 85% of all LVC procedures.

An early practice of LASIK involves making a corneal flap by means of a physical knife (i.e. a microkeratome), lifting the corneal flap, and removing the exposed corneal tissue with laser, thereby altering corneal curvature. More recently, femtosecond laser is used for the same purpose. In such a procedure, tens of thousands of pulsed laser beams generate photodisruption to form micro spot cavitations, which lead to tissue isolation, thereby forming the desired corneal flap. Femtosecond laser, as a newly emerged technology, is safer, more repeatable, more predictable and more flexible as compared to a microkeratome, and therefore has become extensively used in LASIK procedures to form corneal flaps. Additional advantages of using femtosecond laser include reducing potential risks of adverse complications related to a microkeratome, such as iatrogenic keratoconus (associated with a steep cornea), corneal flap dislocation (associated with a flat cornea) and irregular corneal flaps.

However, as a result of either of the foregoing LASIK procedures, the relatively large corneal flap can significantly weaken the strength of the cornea, making the treated cornea vulnerable to the intraocular pressure.

In addition to LASIK procedures, femtosecond laser is also used in the newly developed LVC procedure to form a corneal lenticule in a corneal tissue and then form a small cut face from which the corneal lenticule can be removed (extracted) from the corneal tissue, thereby altering the corneal radian. Such a procedure, by eliminating the need to generate a corneal flap with a large cut face, is less likely to lead to dislocation of the resulting corneal flap and can better maintain the strength of the cornea.

To detail the foregoing procedure, the corneal lenticule is formed by making two cut surfaces in the corneal tissue to be treated using femtosecond laser. The two cut surfaces include one anterior cut face following the profile of the cornea (hereinafter referred to as the Cap) and a posterior cut face having a curvature greater than that of the Cap (hereinafter referred to as the Curvature). Then a small cut face is made at the outer periphery of the corneal lenticule so that the small cut face passes through the outer surface of the cornea and allows the corneal lenticule to be removed from the corneal tissue therethrough. In this way, after the corneal lenticule is removed, the outer curvature of the cornea changes, and the difference is right the diopter required by correction of ametropia (i.e. vision correction).

The disclosure of U.S. patent Ser. No. 10/682,256 is related to removal of a corneal lenticule. Therein, for preventing the corneal lenticule from breakage during its removal from the corneal tissue (particularly at the rim of the corneal lenticule), a minimum thickness of between 5 and 50 μm is provided between the anterior cut face and the posterior cut face that form the corneal lenticule, so as to provide proper strength at the edge of the corneal lenticule. However, as shown in FIGS. 5A and 5B, with the non-zero minimum thickness DH kept between the anterior cut face and the posterior cut face, the thickness of the corneal lenticule to be removed (extracted) is increased, making the resulting cornea thinner and more vulnerable to the intraocular pressure than it would be without having the non-zero minimum thickness kept in the removed corneal lenticule.

### SUMMARY OF THE INVENTION

The objective of the present invention is to provide a method and an apparatus for treating ocular tissue, which can advantageously overcome the problems of the prior art by maintaining sufficient postoperative strength of the treated ocular tissue and preventing the removed (extracted) part of the ocular tissue from breakage.

The disclosed method for treating an ocular tissue comprises generating a femtosecond laser beam from a laser source; orientating the femtosecond laser beam toward the ocular tissue; defining a target area in the ocular tissue using the femtosecond laser beam, wherein the target area contains a sharp-edge part and a to-be-removed part, in which the sharp-edge part has a minimum thickness whose value is gradually reduced to zero, and in which the sharp-edge part is ablated by the femtosecond laser beam while the target area is defined; and removing a to-be-removed parts of the target area from the ocular tissue.

With the disclosed method, since all the sharp-edge parts of the target area are ablated by the femtosecond laser beam before the to-be-removed part of the target area is removed, there is only the to-be-removed part of the target area left to remove from the ocular tissue, and the to-be-removed part is unlikely to have breakage during its removal (extraction) form the ocular tissue.

Additionally, the disclosed method eliminates the need of keeping a non-zero minimum thickness of the part to be removed (extracted) in the ocular tissue as described previously to prevent breakage of the part removed (extracted) from the ocular tissue. The disclosed method can adapt the thickness of the defined target area to practical needs for treatment of the ocular tissue, and the minimal value of this thickness may be down to zero. In other words, the disclosed method eliminates the need of any extra thickness of the target area other than that is exactly required by the treatment (e.g., correction) of the ocular tissue, thereby maximally maintaining postoperative strength of the treated ocular tissue.

The present invention further provides an apparatus for treating an ocular tissue, which comprises a laser source, configured to generate a femtosecond laser beam; an optical system, configured to orientate the femtosecond laser beam generated by the laser source; an optical scanning movement device, configured to focus the femtosecond laser beam from the optical system onto the ocular tissue through a condenser; a controller, connected to the laser source, the optical system, and the optical scanning movement device, and configured to control the laser source to generate the femtosecond laser beam; control the optical system to orientate the femtosecond laser beam toward the optical scanning movement device; and control the optical scanning movement device to define a target area in the ocular tissue using the femtosecond laser beam, wherein the target area includes a sharp-edge part and a to-be-removed part, in which the sharp-edge part has a minimum thickness whose value is gradually reduced to zero, and in which the sharp-edge part is ablated by the femtosecond laser beam while the target area is defined; and a removing device, configured to remove the to-be-removed part of the target area from the ocular tissue in response to operation from a user.

With the disclosed apparatus, since all the sharp-edge parts of the target area are ablated by the femtosecond laser beam under the control of the controller and the optical scanning movement device before the to-be-removed part of the target area is removed, there is only the to-be-removed part of the target area left to remove from the ocular tissue, and the to-be-removed part is unlikely to have breakage during its removal (extraction) form the ocular tissue.

Additionally, the disclosed apparatus eliminates the need of keeping a non-zero minimum thickness of the part to be removed (extracted) in the ocular tissue as described previously using the optical scanning movement device to prevent breakage of the part removed (extracted) from the ocular tissue. The disclosed apparatus can adapt the thickness of the defined target area to practical needs for treatment of the ocular tissue using the controller and the optical scanning movement device, and the minimal value of this thickness may be down to zero. In other words, the disclosed apparatus eliminates the need of any extra thickness of the target area other than that is exactly required by the treatment (e.g., correction) of the ocular tissue, thereby maximally maintaining postoperative strength of the treated ocular tissue.

### BRIEF DESCRIPTION OF THE DRAWINGS

The invention as well as a preferred mode of use, further objectives and advantages thereof will be best understood by reference to the following detailed description of illustrative embodiments when read in conjunction with the accompanying drawings, wherein:

FIG. 1 is a flowchart of a method for treating ocular tissue according to one embodiment of the present invention;

FIG. 2A is a front view of an ocular tissue showing a target area defined using the method in one example of the present invention;

FIG. 2B is a cross-sectional view of the ocular tissue taken along Line II-II of FIG. 2A;

FIG. 3A is front view of an ocular tissue showing a target area defined using the method in another example of the present invention;

FIG. 3B is a cross-sectional view of the ocular tissue taken along Line III-III of FIG. 3A;

FIG. 4 is a block diagram of an apparatus for treating ocular tissue according to one embodiment of the present invention; and

FIGS. 5A and 5B are cross-sectional views illustrating generation of corneal lenticule according to a prior-art method.

### DETAILED DESCRIPTION OF THE INVENTION

For further illustrating the means and functions by which the present invention achieves the certain objectives, the following description, in conjunction with the accompanying drawings and preferred embodiments, is set forth as below to illustrate the implement, structure, features and effects of the subject matter of the present invention. Unless otherwise noted, like elements will be identified by identical numbers throughout all figures and repeated description related thereto is omitted.

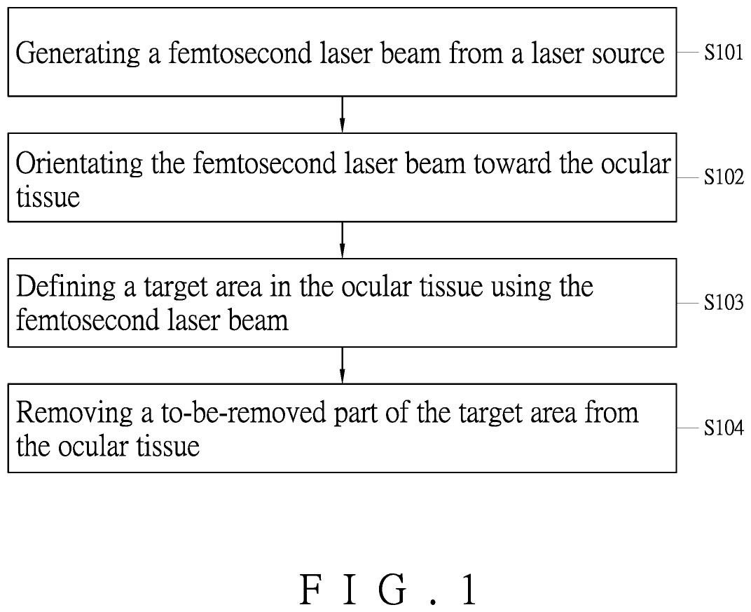

First, please refer to FIG. 1 for a method for treating ocular tissue according to one embodiment of the present invention. In the description below, some embodiments of the present invention will be discussed with reference to a cornea of an eye as the ocular tissue to be treated. However, the use of the disclosed method is not limited thereto. For example, the disclosed method for treating an ocular tissue may be alternatively used for other ocular tissues, such as a lens of an eye.

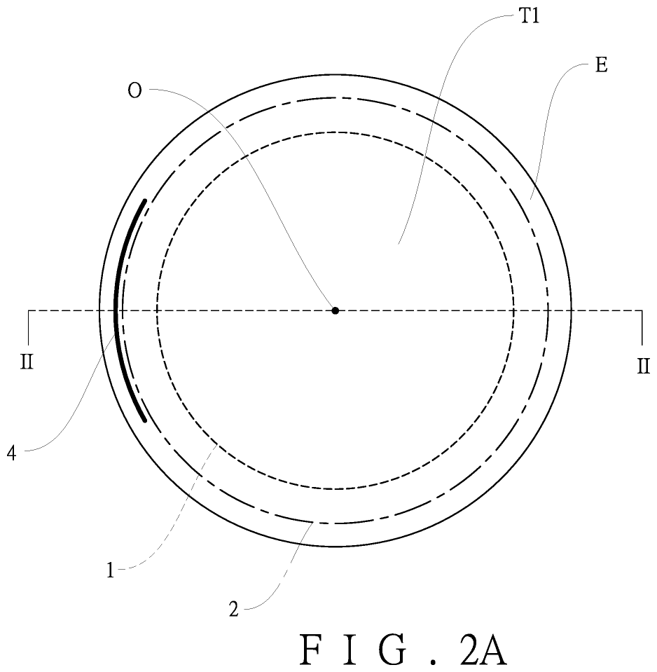

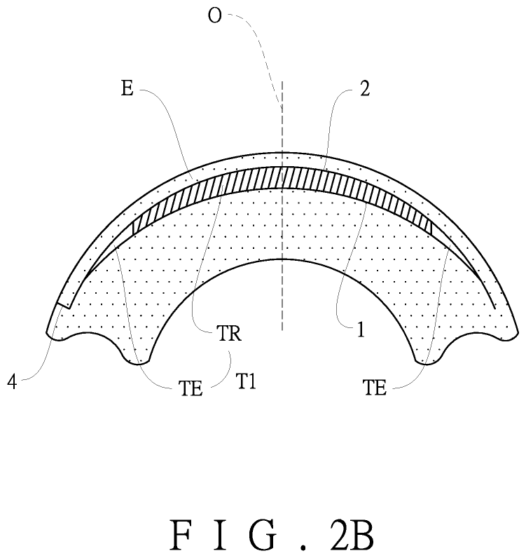

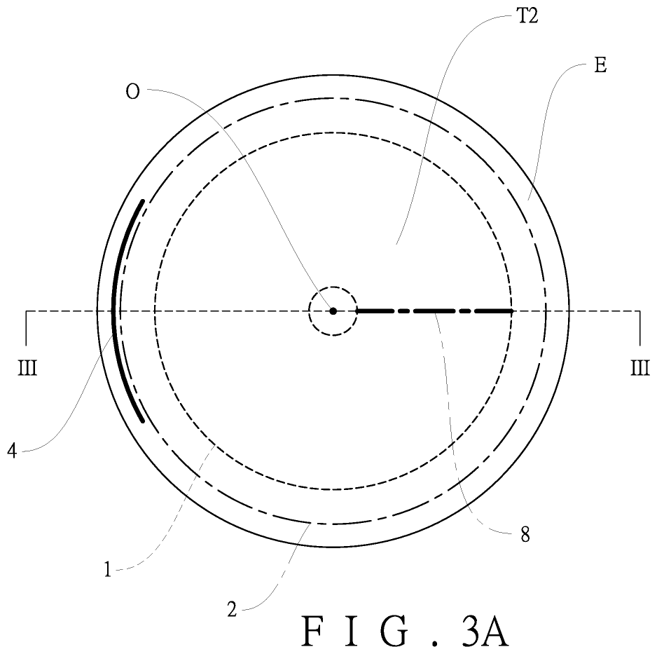

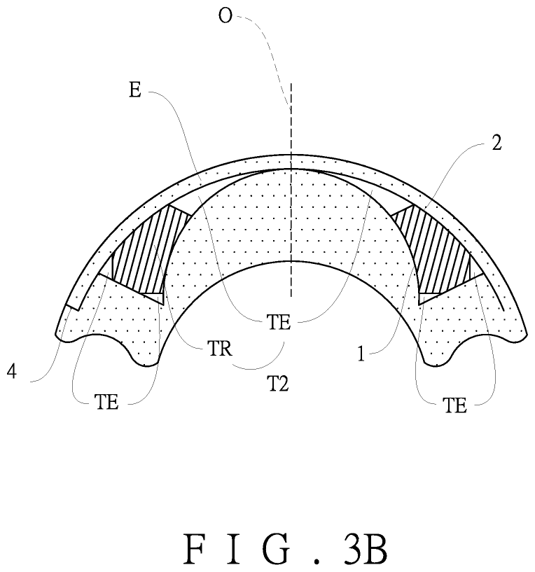

As shown in FIG. 1, the disclosed method for treating an ocular tissue comprises steps S101 through S104. First, the step S101 is for generating a femtosecond laser beam from a laser source. Then the step S102 involves orientating the femtosecond laser beam toward the ocular tissue. Afterward, in the step S103, a target area T1 or T2 is defined in the ocular tissue E using the femtosecond laser beam (referring to FIGS. 2A through 3B). The target area T1 or T2 contains a sharp-edge part TE and a to-be-removed part TR. The sharp-edge part TE may have a minimum thickness whose value is gradually reduced to zero. More details of the target area T1 or T2 will be given below with reference to FIGS. 2A through 3B. At last, in the step S104, the to-be-removed part TR of the target area T1 or T2 is removed from the ocular tissue E.

Two examples of using the disclosed method to treat an ocular tissue E by defining a target area T1 or T2 will be described below with reference to FIGS. 2A through 3B.

In the example shown in FIGS. 2A and 2B, a target area T1 is defined in the ocular tissue E using the disclosed method.

Referring to FIGS. 2A and 2B, defining the target area T1 in the ocular tissue E involves: making a posterior cut face 1 in the ocular tissue E using the femtosecond laser beam, then ablating the sharp-edge part TE of the target area T1 using the femtosecond laser beam, and at last making an anterior cut face 2 in the ocular tissue E that is connected to the posterior cut face 1 using the femtosecond laser beam. The target area T1 such treated has the sharp-edge part TE ablated and cavitalized by the femtosecond laser beam, with merely the to-be-removed part TR left.

In the example shown in FIGS. 2A and 2B, the anterior cut face 2 and posterior cut face 1 formed by the femtosecond laser beam are connected to each other at the outer periphery of the posterior cut face 1. Therefore, the sharp-edge part TE of the target area T1 includes the connection of the anterior cut face 2 and the posterior cut face 1 at the outer periphery of the posterior cut face 1.

In addition, for removing the to-be-removed parts TR of the target area T1 from the ocular tissue E, the disclosed method further comprises making a removal incision 4 in the ocular tissue E using the femtosecond laser beam. The removal incision 4 is located at the outer periphery of the anterior cut face 2 and extends from the outer surface of the ocular tissue E to the anterior cut face 2, which means the removal incision 4 is also connected to the target area T1. In this way, the to-be-removed parts TR of the target area T1 can be removed from the ocular tissue E through the removal incision 4.

After the to-be-removed parts TR of the target area T1 shown in FIGS. 2A and 2B is removed from the ocular tissue E, the outer curvature of the ocular tissue E (i.e. the cornea in the present example) is altered (e.g., flattened), thereby changing the radian of the cornea and achieving desired vision correction. In other words, the target area T1 as shown in FIGS. 2A and 2B is an example of a target area needing to be removed from the cornea for myopia correction.

In the example shown in FIGS. 3A and 3B, a target area T2 is defined in the ocular tissue E using the disclosed method.

Referring to FIGS. 3A and 3B, the target area T2 is defined in the ocular tissue E in a manner similar to that is used to define the target area T1 as shown in FIGS. 2A and 2B with the following difference. To define the target area T2 as shown in FIGS. 3A and 3B, a posterior cut face 1 made using the femtosecond laser beam has a W-like shape. Thus, the anterior cut face 2 also made using the femtosecond laser beam is connected to the posterior cut face 1 not only at the outer periphery of the posterior cut face 1 but also at a site near the central axis O of the target area T2 (referring to FIG. 3B). The sharp-edge part TE of the target area T2 therefore includes the connections between the anterior cut face 2 and the posterior cut face 1 at the outer periphery of the posterior cut face 1, and at the site near the central axis O. More particularly, the sharp-edge part TE of the target area T2 further includes corners at two sides of the W-shaped posterior cut face 1. The sections of the sharp-edge part TE of the target area T2 can all be ablated and cavitalized by the femtosecond laser beam.

In addition, to remove the to-be-removed parts TR of the target area T2 from the ocular tissue E, similar to removing the to-be-removed parts TR of the target area T1 from the ocular tissue E as described above, the disclosed method for treating ocular tissue further comprises making a removal incision 4 in the ocular tissue E using femtosecond laser. The removal incision 4 is located at the outer periphery of the anterior cut face 2 and extends from the outer surface of the ocular tissue E to the anterior cut face 2, so the removal incision 4 is also connected to the target area T2. In this way, the to-be-removed parts TR of the target area T2 may be removed from the ocular tissue E through the removal incision 4.

However, in the example depicted in FIGS. 3A and 3B, since the anterior cut face 2 and the posterior cut face 1 of the target area T2 are connected to each other at both the outer periphery of the posterior cut face 1 and the site near the central axis O (referring to FIG. 3B). When viewed from the viewpoint of FIG. 3A, the target area T2 has a donuts-like shape. In this case, to remove the to-be-removed parts TR of the target area T2 through the removal incision 4, it is necessary to ablate a cut-off surface 8 in the ocular tissue E additionally using femtosecond laser (referring to FIG. 3A). The cut-off surface 8 is connected to the connection between the anterior cut face 2 and the posterior cut face 1 of the target area T2 at the outer periphery of the posterior cut face 1 and at a site near the central axis O, and passes through the target area T2, so that the target area T2 is divided by the cut-off surface 8, making it easy to remove the to-be-removed parts TR of the target area T2 through the removal incision 4.

For further facilitating even removal of the to-be-removed parts TR of the target area T2 through the removal incision 4, the removal incision 4 and the cut-off surface 8 are preferably opposite sides against the central axis O of the target area T2.

After the to-be-removed parts TR of the target area T2 as shown in FIGS. 3A and 3B is removed from the ocular tissue E, the outer curvature of the ocular tissue E (i.e. the cornea in the present example) is altered (e.g., curved), thereby changing the radian of the cornea and achieving desired vision correction. In other words, the target area T1 as shown in FIGS. 3A and 3B is an example of a target area needing to be removed from the cornea for hyperopia correction.

It is clear from the examples of the target area T1 or T2, as shown in FIGS. 2A through 3B, defined using the disclosed method for treating the ocular tissue E that since the sharp-edge part TE of the target area T1 or T2 is ablated by the femtosecond laser beam before the to-be-removed part TR of the target area T1 or T2 is removed, there is only the to-be-removed part TR of the target area T1 or T2 left to remove from the ocular tissue E, and the to-be-removed part TR is unlikely to have breakage during its removal (extraction) form the ocular tissue E. Therefore, the disclosed method is effective in preventing the part (i.e., the target area T1 or T2) to be removed from the ocular tissue (e.g., the cornea) from breakage during its removal.

Moreover, by using the disclosed method, the part of the ocular tissue E (e.g., the cornea) to be removed is unlikely to break when removed. Different from the prior-art solution that keeps a non-zero minimum thickness in the target area to prevent breakage during removal of the part of the ocular tissue, the disclosed method can adapt the thickness of the target area to practical needs for treatment of the ocular tissue and the minimum value of this thickness may be down to zero. In other words, the disclosed method eliminates the need of any extra thickness (e.g., the minimum thickness in the prior art) of the target area other than that is exactly required by the treatment (e.g., correction) of the ocular tissue, thereby maximally maintaining postoperative strength of the treated ocular tissue.

Moreover, by adapting the thickness of the target area to the exact needs of the desired treatment instead of providing a non-zero minimum thickness to the target area, the disclosed method contributes to more accurate decision of the range of the target area in the ocular tissue for the needed treatment (e.g., correction), which in turn provides a wider treatment coverage (i.e. a larger diopter range of correction). For example, for vision correction, assuming that the maximum value of the thickness to be removed that the cornea can withstand is DA, since the prior-art solution required a non-zero minimum thickness DH to be kept in the target area to be removed, the maximum diopter range of correction is a range corresponding to a thickness equal to DA-DH (i.e. subtracting the required non-zero minimum thickness from the maximum value of the thickness to be removed that the cornea can withstand). By comparison, since the disclosed method eliminates the need of keeping a non-zero minimum thickness in the target area, the diopter range of correction it can provide is corresponding to DA, or the maximum value of the thickness to be removed that the cornea can withstand.

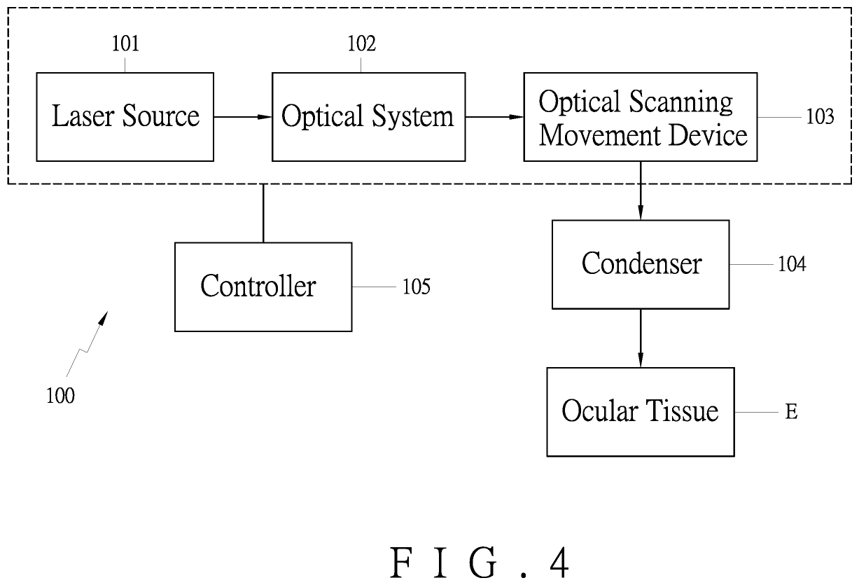

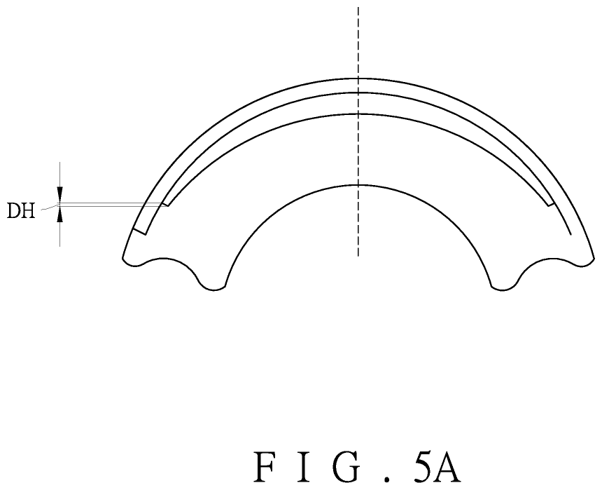

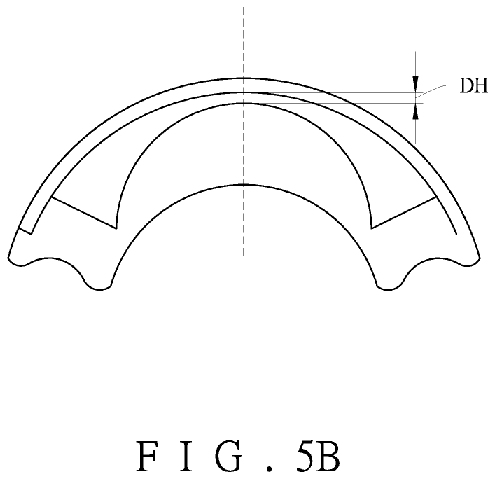

FIG. 4 is a block diagram illustrating an apparatus 100 for treating an ocular tissue according to one embodiment of the present invention.

The apparatus 100 for treating ocular tissue according to one embodiment of the present invention comprises a laser source 101, configured to generating femtosecond laser beam; an optical system 102, configured to orientate the femtosecond laser beam generated by the laser source 101; an optical scanning movement device 103, configured to apply the femtosecond laser beam coming from the optical system 102 to the ocular tissue E through the condenser 104; and a controller 105, connected to the laser source 101, the optical system 102 and the optical scanning movement device 103, and configured to control the laser source 101, the optical system 102 and optical scanning movement device 103, so as to define a target area T1 or T2 in the ocular tissue E (referring to FIGS. 2A through 3B). The apparatus 100 of the present embodiment further comprises a removing device (not shown), which is configured to remove a to-be-removed part TR of the target area T1 or T2 from the ocular tissue E in response to a user's operation (referring to FIGS. 2A through 3B).

Particularly, the controller 105 is configured to control the laser source 101 to generate a femtosecond laser beam, to control the optical system 102 to orientate the femtosecond laser beam toward the optical scanning movement device 103, and to control the optical scanning movement device 103 to focus the femtosecond laser beam on the ocular tissue E through the condenser 104 so as to define the target area T1 or T2. The target area T1 or T2 contains a sharp-edge part TE and a to-be-removed part TR. The sharp-edge part TE may have a minimum thickness whose value is gradually reduced to zero, and the sharp-edge part TE is to be ablated by the femtosecond laser beam while the target area T1 or T2 is defined.

The detail of how the controller 105 defines the target area T1 or T2 in the ocular tissue E is as described previously for FIGS. 2A through 3B and is not repeated herein.

Similarly, by using the disclosed apparatus 100, since the controller 105 controls the optical scanning movement device 103 to ablate the sharp-edge part TE of the target area T1 or T2 using the femtosecond laser beam before the removing device (not shown) removes the to-be-removed part TR of the target area T1 or T2, there is merely the to-be-removed part TR left in the target area T1 or T2 to remove, and the to-be-removed part TR is unlikely to break when the target area T1 or T2 is removed from the remove ocular tissue E. Therefore, the disclosed apparatus is effective in preventing the part (i.e., the target area T1 or T2) to be removed from the ocular tissue (e.g., the cornea) from breakage during its removal.

Moreover, by using the disclosed apparatus 100, the part of the ocular tissue E (e.g., the cornea) to be removed is unlikely to break when removed. Different from the prior-art solution that keeps a non-zero minimum thickness in the target area to prevent breakage during removal of the part of the ocular tissue, the disclosed apparatus can use the controller and the optical scanning movement device to adapt the thickness of the target area to practical needs for treatment of the ocular tissue and the minimum value of this thickness may be down to zero. In other words, the disclosed apparatus eliminates the need of any extra thickness (e.g., the minimum thickness in the prior art) of the target area other than that is exactly required by the treatment (e.g., correction) of the ocular tissue, thereby maximally maintaining postoperative strength of the treated ocular tissue.

Similarly, by adapting the thickness of the target area to the exact needs of the desired treatment instead of keeping a non-zero minimum thickness, the disclosed apparatus 100 contributes to more accurate decision of the range of the target area in the ocular tissue for the needed treatment (e.g., correction), which in turn provides a wider treatment coverage (i.e. a larger diopter range of correction). As this advantage has been detailed above, no repeated description is provided herein.

The accompanying drawings provided herein and referred to by the above description are for easy understanding of the disclosure. The drawings are only exemplificative and may be not made to scale, which means some features may be exaggerated while others may be understated. Thus, the drawings shall be deemed to be illustrative but not limiting.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims

What is claimed is:

1. A method for treating an ocular tissue, comprising:
generating a femtosecond laser beam from a laser source;
orientating the femtosecond laser beam toward the ocular tissue;
defining a target area in the ocular tissue using the femtosecond laser beam, wherein the target area contains a sharp-edge part and a to-be-removed part, in which the sharp-edge part has a minimum thickness whose value is gradually reduced to zero, and in which the sharp-edge part is ablated by the femtosecond laser beam while the target area is defined; and
removing the to-be-removed part of the target area from the ocular tissue.

2. The method of claim 1, further comprising: before the to-be-removed part of the target area is removed from the ocular tissue, making a removal incision in the ocular tissue using the femtosecond laser beam,
wherein the removal incision extends from an outer surface of the ocular tissue to the target area, and
wherein the to-be-removed part of the target area is removed from the ocular tissue through the removal incision.

3. The method of claim 1, wherein defining the target area in the ocular tissue using the femtosecond laser beam further comprises:
making a posterior cut face in the ocular tissue;
ablating the sharp-edge part of the target area using the femtosecond laser beam; and
making an anterior cut face in the ocular tissue so that the anterior cut face is connected to the posterior cut face.

4. The method of claim 3, wherein the anterior cut face and the posterior cut face are connected to each other at an outer periphery of the posterior cut face; and
wherein the sharp-edge part of the target area includes a connection between the anterior cut face and the posterior cut face at the outer periphery of the posterior cut face.

5. The method of claim 4, further comprising:
before the to-be-removed part of the target area is removed from the ocular tissue, making a removal incision in the ocular tissue using the femtosecond laser beam,
wherein the removal incision extends from an outer surface of the ocular tissue to the target area, and
wherein the to-be-removed part of the target area is removed from the ocular tissue through the removal incision.

6. The method of claim 3, wherein the anterior cut face and the posterior cut face are connected to each other at an outer periphery the posterior cut face and at a site near a central axis of the target area;
in which the sharp-edge part of the target area includes connections between the anterior cut face and the posterior cut face at the outer periphery of the posterior cut face and at the site near the central axis; and
in which defining the target area in the ocular tissue using the femtosecond laser beam further comprises ablating a cut-off surface that is connected to the connections between the anterior cut face and the posterior cut face at the outer periphery of the posterior cut face and at the site near the central axis, and passes through the target area.

7. The method of claim 6, further comprising:
before the to-be-removed part of the target area is removed from the ocular tissue, making a removal incision in the ocular tissue using the femtosecond laser beam,
wherein the removal incision extends from an outer surface of the ocular tissue to the target area, and
wherein the to-be-removed part of the target area is removed from the ocular tissue through the removal incision.

8. The method of claim 7, wherein the cut-off surface and the removal incision are opposite sides against the central axis of the target area.

9. The method of claim 1, wherein the ocular tissue is a cornea or a lens of an eye.

10. An apparatus for treating an ocular tissue, comprising:
a laser source, configured to generate a femtosecond laser beam;
an optical system, configured to orientate the femtosecond laser beam generated by the laser source;
an optical scanning movement device, configured to focus the femtosecond laser beam from the optical system onto the ocular tissue through a condenser;
a controller, connected to the laser source, the optical system, and the optical scanning movement device, and configured to control the laser source to generate the femtosecond laser beam;
control the optical system to orientate the femtosecond laser beam toward the optical scanning movement device; and
control the optical scanning movement device to define a target area in the ocular tissue using the femtosecond laser beam, wherein the target area includes a sharp-edge part and a to-be-removed part, in which the sharp-edge part has a minimum thickness whose value is gradually reduced to zero, and in which the sharp-edge part is ablated by the femtosecond laser beam while the target area is defined; and
a removing device, configured to remove the to-be-removed part of the target area from the ocular tissue in response to operation from a user.

11. The apparatus of claim 10, wherein the controller is further configured to control the optical scanning movement device to make a removal incision in the ocular tissue that extends from an outer surface of the ocular tissue to the target area using the femtosecond laser beam before the to-be-removed part of the target area is removed from the ocular tissue by the removing device, and
wherein the removing device is configured to remove the to-be-removed part of the target area from the ocular tissue through the removal incision in response to the operation from the user.

12. The apparatus of claim 10, wherein that the controller controls the optical scanning movement device to define the target area in the ocular tissue using the femtosecond laser beam comprises:
making a posterior cut face in the ocular tissue;
ablating the sharp-edge part of the target area; and
making an anterior cut face in the ocular tissue so that the anterior cut face is connected to the posterior cut face.

13. The apparatus of claim 12, wherein the anterior cut face and the posterior cut face are connected at an outer periphery of the posterior cut face; and
wherein the sharp-edge part of the target area includes a connection between the anterior cut face and the posterior cut face at the outer periphery of the posterior cut face.

14. The apparatus for treating ocular tissue of claim 13, wherein the controller is further configured to control the optical scanning movement device to make a removal incision in the ocular tissue that extends from an outer surface of the ocular tissue to the target area using the femtosecond laser beam before the to-be-removed part of the target area is removed from the ocular tissue by the removing device, and
wherein the removing device is configured to remove the to-be-removed part of the target area from the ocular tissue through the removal incision in response to the operation from the user.

15. The apparatus of claim 12, wherein the anterior cut face and the posterior cut face are connected at an outer periphery of the posterior cut face and at a site near a central axis of the target area;
in which the sharp-edge part of the target area includes the connections between the anterior cut face and the posterior cut face at the outer periphery of the posterior cut face and at the site near the central axis; and
in which that the controller controls the optical scanning movement device to define the target area in the ocular tissue using the femtosecond laser beam further comprises ablating a cut-off surface that is connected to the connections between the anterior cut face and the posterior cut face at the outer periphery of the posterior cut face and at the site near the central axis, and passes through the target area.

16. The apparatus of claim 15, wherein the controller is further configured to control the optical scanning movement device to make a removal incision in the ocular tissue that extends from an outer surface of the ocular tissue to the target area using the femtosecond laser beam before the removing device removes the to-be-removed part of the target area from the ocular tissue, and
wherein the removing device is configured to remove the to-be-removed part of the target area from the ocular tissue through the removal incision in response to the operation from the user.

17. The apparatus of claim 16, wherein the cut-off surface and the removal incision are opposite sides against the central axis of the target area.

18. The apparatus of claim 10, wherein the ocular tissue is a cornea or a lens of an eye.
